(19) United States
(12) Patent Application Publication
(10) Pub. No.: US 2011/0270281 A1
(43) Pub. Date: Nov. 3, 2011

(54) Articulating Axial Needle Grasper

(75) Inventor: Jaroslaw T. Malkowski, Trumbull, CT (US)

(73) Assignee: Tyco Healthcare Group LP, New Haven, CT (US)

(21) Appl. No.: 13/070,669

(22) Filed: Mar. 24, 2011

Related U.S. Application Data
(60) Provisional application No. 61/329,595, filed on Apr. 30, 2010.

(51) Int. Cl. A61B 17/04

(52) U.S. Cl. 606/147

(57) ABSTRACT

An end effector is presented having an elongate body portion having a proximal end and a distal end. The end effector also includes a rotating head configured to be disposed at the distal end of the body portion and an actuation member configured to be disposed at the proximal end of the body portion. The actuation member is in operative cooperation with a handle assembly to enable rotational movement of the rotating head. The rotating head further cooperates with a needle movable relative to the rotating head.

### CROSS-REFERENCE TO RELATED APPLICATIONS

This application claims the benefit of and priority to U.S. Provisional Patent Application No. 61/329,595, filed Apr. 30, 2010, the entire disclosure of which is incorporated by reference herein.

### BACKGROUND

1. Technical Field

The present disclosure relates to endoscopic and laparoscopic surgical devices and, more particularly, to a laparoscopic surgical device including an articulating axial needle grasper.

2. Background of Related Art

As an alternative to open surgical procedures, many surgeons use endoscopic or laparoscopic instruments for remotely accessing organs through smaller, puncture-like incisions or natural orifices. As a direct result thereof, patients tend to benefit from less scarring and reduced healing time.

Endoscopic instruments, for example, are inserted into the patient through a cannula, or port, which has been made with a trocar. Typical sizes for cannulas range from three millimeters to twelve millimeters. Smaller cannulas are usually preferred, which, as can be appreciated, ultimately presents a design challenge to instrument manufacturers who must find ways to make endoscopic instruments that fit through the smaller cannulas.

Often while utilizing a suture and needle combination, it is difficult to grasp and pull the entire needle through the tissue. This common situation is usually due to the suture needle being too large for the space provided by a particular incision site. In addition to the limited space available by incision sites, the size, shape, and/or rigidity of the suture needle utilized, may be self-limiting during the surgical procedure. Additionally, one may be unable to pull the needle through tissues due to the proximity of anatomical structures which are at risk of injury caused by the suture needle. Therefore, there is a need to provide for a needle assembly that allows a surgeon to quickly and effectively suture tissue.

### SUMMARY

In accordance with the present disclosure, an end effector is provided. The end effector may have an elongate body portion having a proximal end and a distal end. The end effector also includes a rotating head configured to be disposed at the distal end of the body portion and an actuation member configured to be disposed at the proximal end of the body portion. The actuation member may be in operative cooperation with a handle assembly to enable rotational movement of the rotating head. The rotating head further cooperates with a needle movable relative to the rotating head.

The end effector further includes a needle that may be curved. The needle may be adapted to receive a suture for suturing tissue. The suture may be a monofilament or a multifilament suture.

The end effector may further include a needle positioned between the rotating head and an axial gripper portion via a needle carrier. The needle may also be removed from the end effector via the needle carrier.

The end effector may also include a body portion that may be flexible or bendable. The bendable body portion may define an angle of curvature ranging from about 0° to about 180°. The body portion may have a diameter of about 0.22 inches. Additionally, the body portion may include one or more links positioned or embedded therein or extending therethrough for enabling a push/pull action. The push/pull action enabled by the one or more links may cause/create the rotational movement of the rotating head.

The rotating head of the end effector may enable stabilization and manipulation of the needle relative to the body portion during a stitching process. The manipulation of the needle may include at least griping, rotating, and/or articulating the needle.

The end effector may be used during minimally invasive procedures.

In accordance with the present disclosure, an axial gripper assembly may be provided. The axial gripper assembly may include a flexible, elongate body portion having a proximal end and a distal end and a rotating gripper head configured to be disposed at the distal end of the body portion. The axial gripper assembly may also include an actuation member configured to be disposed at the proximal end of the body portion, the actuation member configured to enable rotational movement of the rotating head. The axial gripper assembly may also include a curved needle positioned between the gripper head and the rotating head via a needle carrier, the curved needle being directionally manipulated in accordance with movement of the rotating head.

In accordance with the present disclosure, a method of suturing may be provided. The method may provide for an axial gripper assembly that has a flexible, elongate body portion having a proximal end and a distal end; a gripper head and a rotating head configured to be disposed at the distal end of the body portion; an actuation member configured to be disposed at the proximal end of the body portion, the actuation member configured to enable rotational movement of the rotating head; and a curved needle positioned between the rotating head and the gripper head via a needle carrier, the curved needle being directionally manipulated in accordance with movement of the rotating head.

### BRIEF DESCRIPTION OF THE DRAWINGS

Various embodiments of the presently disclosed end effector assembly are described hereinbelow with references to the drawings, wherein:

FIG. 1 is a perspective view of an end effector having a rotating head and a curved needle, in accordance with the present disclosure;

FIG. 2 is a front end view of the end effector having the rotating head and the curved needle of FIG. 1, where the needle pierces the tissue, in accordance with the present disclosure;

FIG. 3 is a front end view of the end effector having the rotating head and the curved needle of FIG. 1, where the suture enters the tissue to seal the cut, in accordance with the present disclosure;

FIG. 4 is a front perspective view of the end effector of FIG. 1, illustrating a needle carrier used for inserting and removing the curved needle from the end effector, in accordance with the present disclosure;

FIG. 5 is a side perspective view of the end effector having the rotating head and the curved needle of FIG. 1, where rotation of the body portion creates rotational movement of the rotating head, which in turn rotates the curved needle, in accordance with the present disclosure;

FIG. 5A is a cross-sectional view of the distal end of the body portion, in accordance with the present disclosure; and

FIG. 6 is a first perspective view, a second end view, and a third end view of the end effector having the rotating head and the curved needle of FIG. 1, illustrating the end effector in operation, in accordance with the present disclosure.

Further scope of applicability of the present disclosure will become apparent from the detailed description given hereinafter. However, it should be understood that the detailed description and specific examples, while indicating preferred embodiments of the present disclosure, are given by way of illustration only, since various changes and modifications within the spirit and scope of the present disclosure will become apparent to those skilled in the art from this detailed description.

### DETAILED DESCRIPTION OF THE EMBODIMENTS

In the figures and in the description that follows, in which like reference numerals identify similar or identical elements, the term “proximal” will refer to the end of the apparatus which is closest to the operator during use, while the term “distal” will refer to the end which is farthest from the operator, as is traditional.

Prior to describing the present disclosure in further detail, it will first be helpful to define various terms that will be used throughout the following discussion. For example:

The term “connect” or “connecting” may refer to adhere, affix, anchor, attach, band, bind, bolt, bond, brace, button, cohere, fasten, couple, embed, establish, fix, grip, hold, hook, implant, link, lock, lodge, screw, seal, rivet, tack on, tighten, or unite. The term “connect” or “connecting” may refer to linking/fastening/attaching/locking any type of materials or elements or components or units in a removable/detachable/interchangeable manner.

In the present disclosure, the term “suture needle” may refer to a needle specifically designed for penetrating bodily tissue and pulling through the tissue a length of suture material to approximate edges of tissue, such as result from an incision or wound, to permit the tissue to join together during healing Suture needles may be severed from the suture material once the suturing procedure of placing sufficient stitches or loops of suture material in the tissue may be completed and are commonly made of metal. That is, suture needles are not designed to remain in the tissue but rather serve only the purpose of penetrating the tissue to position the suture material therein. After the suturing procedure and severing of the suture needles, the suture needles may conventionally be sterilized for reuse or, more commonly, discarded.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

Embodiments will be described below while referencing the accompanying figures. The accompanying figures are merely examples and are not intended to limit the scope of the present disclosure.

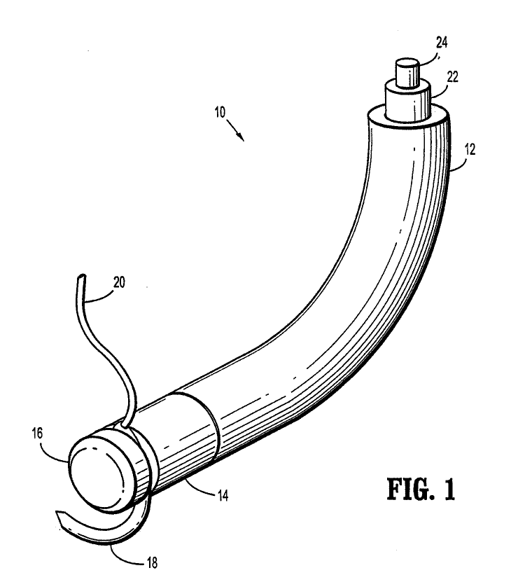

Referring to FIGS. 1-6, end effector 10 generally includes a body portion 12 having a proximal end and a distal end. The proximal end of the body portion 12 may be connected to an actuation member (not shown) being in operative cooperation with a handle assembly (not shown). The handle assembly may be in cooperation with the actuation member and may enable rotational movement of a rotating head 14. The distal end of the body portion 12 may include the rotating head 14. The rotating head 14 may cooperate with an axial gripper 16. Positioned between the rotating head 14 and the axial gripper 16 may be a needle 18. Of course, one skilled in the art may contemplate placing the needle 18 in any type of configuration in relation to the body portion 12 and the rotating head 14. One end of the needle 18 may include a suture 20. The body portion 12 may include a rotating shield or torque coil 22 in operable communication with a push/pull connecting member 24. Connecting member 24 may be a cable, a link, a plurality of links, a rod, etc. One skilled in the art may contemplate using a plurality of different connecting members.

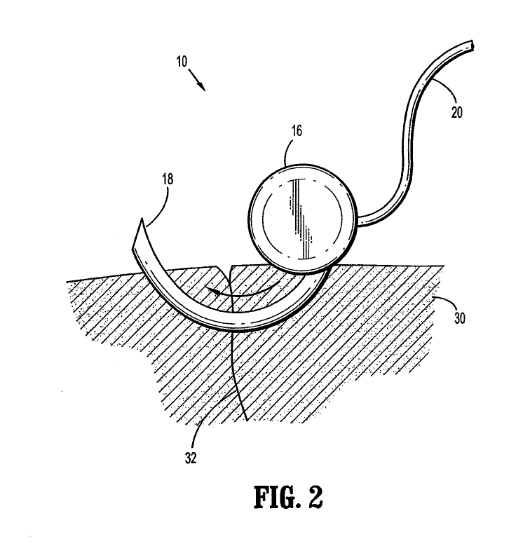

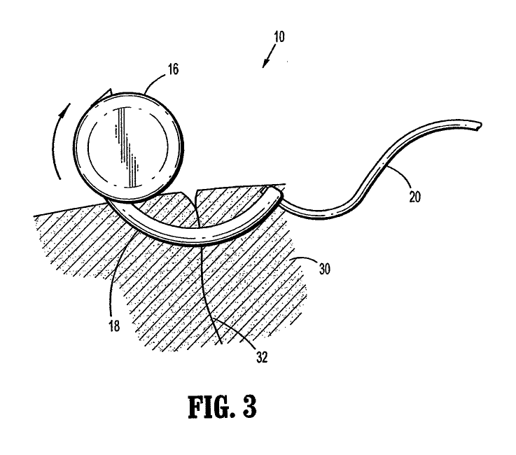

The needle 18 of the end effector 10 may be a curved needle. However, it is contemplated that the needle 18 may be of any shape or size and constructed of any type of suitable material. The needle 18 may be adapted to receive a suture 20 for suturing, for example, tissue 30 (see FIGS. 2 and 3). The suture 20 may be a monofilament or a multifilament suture. Suture material may be classified as either absorbable or non-absorbable. Absorbable suture may be placed below the skin surface where in time, the body decomposes, dissolves, and absorbs the suture material. There are numerous non-absorbable suture materials also used during surgical procedures. The non-absorbable materials may be employed and manually removed after the intended purpose has been completed such as a surgical site that is considered healed.

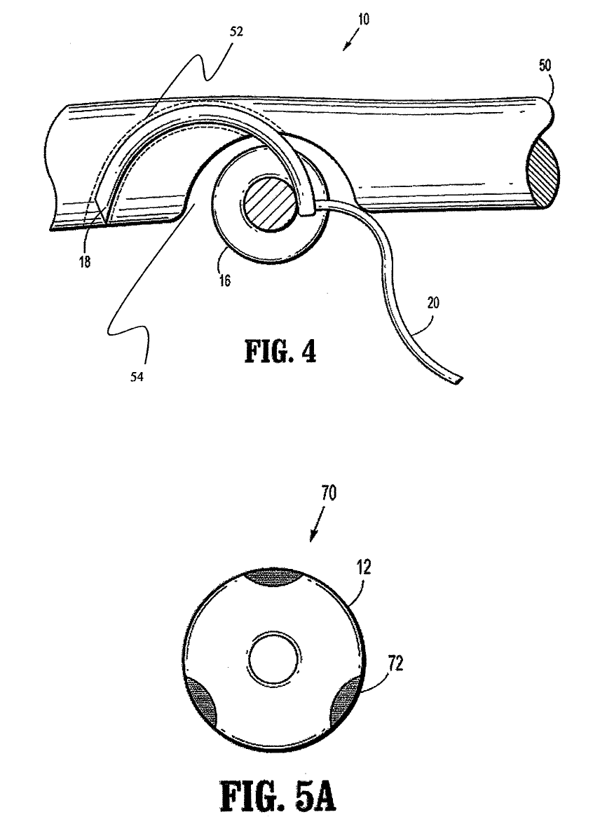

The needle 18 may be positioned between the rotating head 14 and the body portion 12 via a needle carrier 50 (see FIG. 4). The needle 18 may be inserted and/or removed via the needle carrier 50. The needle carrier 50 may be any type of instrument for positioning a needle at a worksite, such as a surgical worksite including one or more different surgical instruments. Further, referring to FIG. 4, the needle carrier 50 may have the same diameter as the insertion size (e.g., 5 mm, 10 mm, etc.). Thus, the needle carrier 50 has at least one cutout 54. Moreover, the needle 18 may be positioned within a channel 52 for holding the needle 18 when inserting or retracting the needle 18 using needle carrier 50. The channel 52 is depicted with phantom lines in FIG. 4.

Further relating to needle 18, suture and needle combinations are commonly used in surgical procedures. The needle 18 may be constructed of stainless steel and may vary in length and shape. The method of attachment of suture 20 to the needle 18 may vary, but the vast majority involves pinching a small length of suture 20 within a small recess on the dull end of needle 18. The needle 18 may be pushed through various tissues 30 (see FIGS. 2 and 3) along with suture 20 trailing within the same path. A major issue presented by conventional suture needle combinations may be the risk of injury to local tissues and anatomical structures during use. FIGS. 1-6 show a suture needle combination that provides a solution to reduce injury to local tissues and anatomical structures. Needle suture combinations as illustrated in FIGS. 1-6 may be of any length and have curves of any number of angles.

The lengths of suture materials 20 may be made of conventional non-bioabsorbable materials or of bioabsorbable materials and may be elastic or stretchable for specific surgical procedures. As shown in FIGS. 1-6, multiple lengths of suture materials 20 may be attached to the suture materials 20 for specific surgical procedures and to affect a knot tying function. The components of the suturing instrument 10 of the present disclosure may be constructed of any suitable, medical grade materials to permit sterilization for reuse or disposal for single patient use. The components may be made of multiple parts of various configurations and materials to reduce cost.

The body portion 12 may be flexible or bendable. The bendable body portion 12 may define an angle of curvature ranging from about 0° to about 180°. The body portion 12 may have a diameter of 0.22 inches. Of course, one skilled in the art may contemplate any type of diameter suitable for any type of application of the end effector/instrument 10.

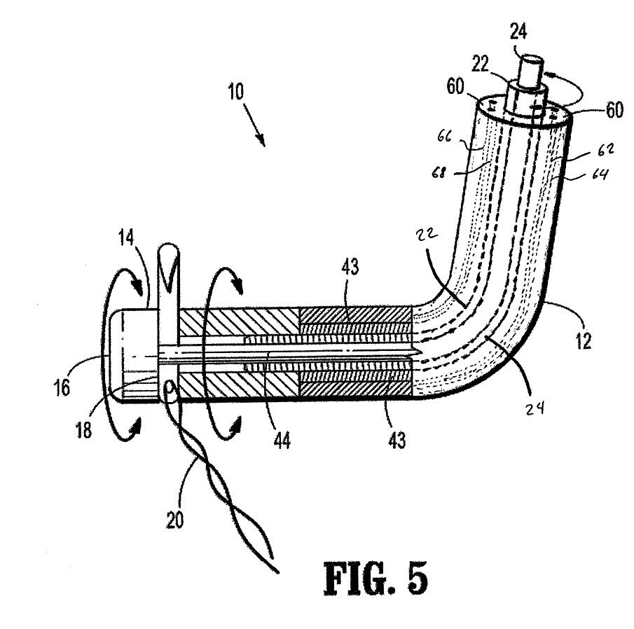

The body portion 12 may include one or more connecting members 24 positioned or embedded therein or extending therethrough for enabling a push/pull action to occur. The push/pull action that may be enabled by the one or more connecting members 24 results in opening/closing rotating head 14 for release or capture of needle 18. The torque coil 22 may rotate clockwise and/or counterclockwise thereby rotating needle 18 for suturing. Therefore, the one or more connecting members 24 rotate as a consequence of the rotation of the torque coil 22. The full extent of the rotating shield 22 and the one or more connecting members 24 will be further described below with reference to FIG. 5.

The rotating head 14 may enable stabilization and precise manipulation of the needle 18 relative to the body portion 12 during a stitching process. The precise manipulation of the tissue 30 may include at least griping, rotating, and/or articulating of the needle 18. Thus, the axial gripper 16 and the rotating head 14 may be activated by the flexible, bendable body portion 12 via the push/pull action of the one or more connecting members 24 located within the rotating shield 22 extending the length of the body portion 12. The rotary or rotational motion may be activated by the actuation member (not shown) in cooperation with the handle assembly (not shown). The handle assembly may include one or more buttons and/or knobs or any other type of actuation mechanism and/or configuration for causing/creating the rotational movement of the end effector 10. The rotational motion may be directly controlled by the one or more push/pull connecting members 24.

Thus, the present disclosure may relate to a surgical suturing device 10, and more specifically, to an ergonomic rotational needle driver 10 which may enhance the tissue suturing procedure, particularly the one performed on restricted, deep and less accessible locations, by incorporating the push/pull connecting members 24 that may prevent issues associated with loss of needle control during a suturing procedure. The embodiments of the present disclosure may also enhance the control surgeons have over the suturing needle 18 by enabling a rotational movement while driving the suturing needle 18 through the tissue 30 that permits to place the needle 18 in the appropriate location in order to continue the subsequent steps of the suturing cycle.

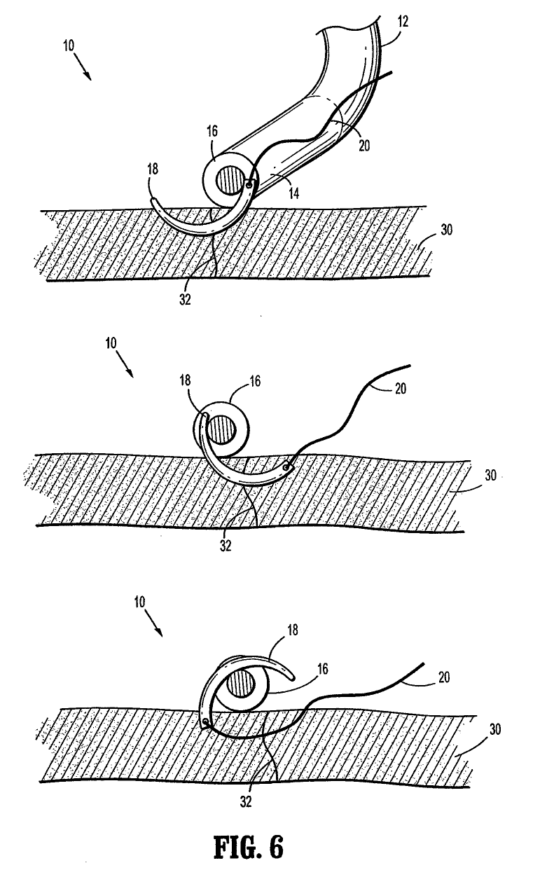

FIGS. 2, 3, and 6 illustrate the operation of the end effector 10. In FIG. 2, the axial gripper 16 may be positioned adjacent tissue 30. A cut 32 may be performed by a surgeon on the tissue 30 during a surgical procedure. The curved needle 18 of the end effector 10 may enter the tissue 30 at the cut 32. At this point, the suture 20 remains outside the tissue 30. In FIG. 3, as the rotating head 14 rotates in a clockwise direction, the needle 18 may be fully inserted into the tissue 30 through the cut 32. At this point, the suture 30 has been inserted into the tissue 30. The suture 20 aims to seal the cut 32 of the tissue 30. FIG. 6 further illustrates how the needle 18 may enter the tissue 30 at the cut 32 in a rotational motion, that may be either a clockwise or a counterclockwise motion, which may be controlled by the surgeon via the push/pull connecting member 24 connected to the actuation member and handle assembly.

With reference to FIG. 5, the use of one or more connecting members 24 that rotate while grasping the needle 18 enables the instrument 10 to perform suturing tasks automatically. Additionally, one or more cables 60 may be positioned within the shaft 44 for articulating the distal end relative to the shaft 44. For example, FIG. 5 illustrates four (4) cables 60. Of course, one skilled in the art may contemplate using a plurality of different cables 60. The cables 60 may also be referred to as articulation cables since they cause articulation movement of the instrument 10. The cables 60 may extend the entire length of the body portion 12, as depicted by phantom lines 62, 64, 66, and 68.

One of the features of the cables 60 may be the ability to drive the needle 18 continuously through the tissue 30 without the need to release and re-drive the needle 18. Once the needle 18 has been rotated to a favorable position it may be grasped at the other side of the free tissue edge. The needle 18 may even be repositioned again, if desired, by rolling the graspers or the surgeon may proceed directly to the other tissue edge. Thus, there is no need to release, re-grasp and re-drive the needle 18. The process may be repeated in the same manner and a suturing cycle may be completed.

In addition, this feature decreases the amount of time, effort and eye-hand coordination that the surgeon invests in performing these tasks. This is especially true when small needles need to be used, such as in a minimally invasive procedure. Therefore, the instrument 10 affords the surgeon the capability of driving the needle 18 with minimal motion of the hand. In deep tissues this translates to more efficient and precise handling of the needle 18. Such a capability may be desired when vascular structures are in the vicinity of the area to be sutured. Reducing wrist rotation may also reduce any potential interference with the line of sight when the operative spaces are very small or narrow, as in the brain. Moreover, the example embodiments may further relate to a laparoscopic gripping device 10 for a surgical needle 18 to be used during a minimally invasive procedure that requires suturing or stitching. The axial gripper head 10 may have the ability to laparoscopically hold and precisely manipulate a surgical needle 18 during the stitching process. The end effector 10 may operate by gripping, holding, rotating, and/or articulating a needle 18 while stitching.

FIG. 5 further illustrates a shaft 44 that may be positioned in the rotating head 14 and the body portion 12 for stabilizing the rotating head 14 in order to control the movement of the curved needle 18. Several structural elements may be positioned within the rotating head 14 and the body portion 12 to aid in the stabilization of the rotating head 14. For example, bushings 43 may be positioned adjacent the shaft 44 to aid in the stabilization of shaft 44. Additionally, referring to FIG. 5A, a cross-sectional view 70 of the distal end of the body portion 12 is illustrated. The cross-sectional view 70 depicts 3 raised areas 72 positioned on an inner surface of the body portion 12. The raised areas 72 aid in holding the needle 18 in an appropriate position. Of course, one skilled in the art may contemplate using a number of different raised areas 72 along the inner surface of the body portion 12, the number of different raised areas 72 constructed into a plurality of different shapes and sizes.

Additionally, the instrument 10 may be locked and unlocked with the same unidirectional motion. As a result, the surgeon does not have to move his hand or fingers any differently when locking or unlocking the instrument 10. This feature may reduce the amount of movements and energy expenditure. Thus, the surgeon's hand experiences less fatigue.

The design described in the example embodiments of the present disclosure may permit rotation of a curved surgical needle 18 without requiring a rotational motion of the wrist. Therefore, the instrument 10 may be held in a position that may not allow wrist rotation and still carry out its function. In some situations the structures to be sutured lie very deep. The surgeon does not need to position his forearm vertically in order to rotate the instrument 10. There may be an advantage when using this instrument 10 that goes beyond any individual benefit granted by its design features. The fact that suturing may become a more streamlined process permits a more continuous flow of the procedure. The surgeon does not have to stop as often to think what he needs to do next. He does not need to refocus on which segment of the tissue to grasp after looking away from the tissue to reposition the needle 18. The end result may be a procedure that is faster, more energy-efficient and safer for both the patient and the surgeon.

In summary, the disclosed embodiments of the present disclosure may help provide an effective suturing device that enhances the maneuvering and safety of suturing procedures. The present disclosure may include a suturing needle driver that comprises an ergonomical handle that eases the suturing process to right and left handed users. It also may include a locking mechanism that may permit users to maintain the needle tightly fixed to the needle driver in order to have a best control over the needle and the movements related to the suturing process.

Additionally, the rotational knob may provide additional control over the movements related to the suturing procedure. The example embodiments of the present disclosure may permit the user to position the suturing needle at the exact angle at which the suturing material has to be inserted into the tissue. The present disclosure may also provide a surgical suturing needle driver that may permit a surgeon to grasp, secure and rotate a curved surgical needle without requiring a rotational motion at the surgeon's wrist.

Some of the advantages of using the embodiments of the present disclosure include the ability of the curved needle to allow physicians to work in smaller incision sites and the reduction of injury to local tissue and the decrease risk to vital structures within or near surgical site while performing procedures. This suture and needle combination also provides a lower cost alternative to using expensive hardware, surgical screws, and other implants for utilized in the surgical procedures. The present disclosure also reduces the risk of infection through smaller surgical openings while in addition to decrease exposure due to reduced operating time needed when performing procedures. The present disclosure further provides a method for having the ability to utilize re-useable suture needle tips, again reducing costs even further.

While the present disclosure has been explained by a detailed description of certain specific embodiments and has focused on medicine (human and veterinary) applications, there are many other industries including upholstery, shoemaking, and tailors (clothing repair) for example, which also may benefit from the example embodiments of the present disclosure. It is understood that in other areas of industry, various modifications and substitutions may be made. These variations should be included within the scope of the appended claims, and also should include the equivalents of such embodiments.

The suture devices of the present disclosure may be any size from micro to macro dependent upon the surgical procedures for which they are designed for use and, it should be appreciated that, while the suture devices of the present disclosure are particularly designed for use in endoscopic or closed procedures, they may also be used in open procedures since the time required for suturing is substantially reduced resulting in a significant reduction in overall operating time. The suture needles may taper throughout their length to a sharp tip or may have a constant diameter or cross section along their length with a sharp conical, pyramidal or polygonal tip at the distal end.

The configuration of the suture needles in cross section may be varied in accordance with surgical procedures to be performed including, for example, circular, semi-circular, oval, lunar, rectangular, hexagonal, and polygonal solid or hollow configurations. Additionally, the outer surfaces of the suture needles may be grooved to facilitate penetration. The suture needles may be made in any conventional manner of working with plastic materials including molding, extrusion, stamping or cutting, and the suture needles and suture materials may be formed simultaneously or separately. Of course, one skilled in the art may contemplate a number of different materials and manufacturing processes for achieving the desired effects.

It will be understood that there are to be no limitations as to the dimensions and shape of the needle, including the body of the axial needle grasper, or the materials from which the needle and body of the axial needle grasper is manufactured or the electronics that may be used to run such a needle assembly. It is to be realized that the optimum dimensional relationships for the parts of the present disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present disclosure.

From the foregoing and with reference to the various figure drawings, those skilled in the art will appreciate that certain modifications may also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An end effector comprising:
an elongate body portion having a proximal end and a distal end;
a rotating head configured to be disposed at the distal end of the body portion; and
an actuation member configured to be disposed at the proximal end of the body portion, the actuation member being in operative cooperation with a handle assembly to enable rotational movement of the rotating head;
wherein the rotating head cooperates with a needle movable relative to the rotating head.

2. The end effector according to claim 1, wherein the needle is curved.

3. The end effector according to claim 1, wherein the needle is adapted to receive a suture for suturing tissue.

4. The end effector according to claim 3, wherein the suture is a monofilament or a multifilament suture.

5. The end effector according to claim 1, wherein the needle is positioned between the rotating head and an axial gripper portion via a needle carrier.

6. The end effector according to claim 1, wherein the needle is removed from the end effector via a needle carrier.

7. The end effector according to claim 1, wherein the body portion is bendable.

8. The end effector according to claim 7, wherein the bendable body portion defines an angle of curvature ranging from about 0° to about 180°.

9. The end effector according to claim 1, wherein the body portion has a diameter of about 0.22 inches.

10. The end effector according to claim 1, wherein the body portion includes one or more links extending therethrough for enabling a push/pull action.

11. The end effector according to claim 10, wherein the push/pull action enabled by the one or more links creates the rotational movement of the rotating head.

12. The end effector according to claim 1, wherein the rotating head enables stabilization and manipulation of the needle relative to the body portion during a stitching process.

13. The end effector according to claim 12, wherein manipulation of the needle includes at least griping, rotating, and/or articulating the needle.

14. The end effector according to claim 1, wherein the end effector is used during minimally invasive procedures.

15. An axial gripper assembly comprising:
a flexible, elongate body portion having a proximal end and a distal end;
a gripper head and a rotating head configured to be disposed at the distal end of the body portion;
an actuation member configured to be disposed at the proximal end of the body portion, the actuation member configured to enable rotational movement of the rotating head; and
a curved needle positioned between the rotating head and the gripper head via a needle carrier, the curved needle being directionally manipulated in accordance with movement of the rotating head.

16. The axial gripper assembly according to claim 15, wherein the curved needle is adapted to receive a suture for performing minimally invasive procedures.

17. The axial gripper assembly according to claim 15, wherein the body portion includes one or more links extending therethrough for enabling a push/pull action that causes the rotational movement of the rotating head.

18. The axial gripper assembly according to claim 15, wherein the gripper head and the rotating head enable stabilization and manipulation of the curved needle relative to the body portion, the manipulation of the curved needle including at least griping, rotating, and/or articulating the needle.

19. A method of suturing, the method comprising:
providing an axial gripper assembly including:
a flexible, elongate body portion having a proximal end and a distal end;
a gripper head and a rotating head configured to be disposed at the distal end of the body portion;
an actuation member configured to be disposed at the proximal end of the body portion, the actuation member configured to enable rotational movement of the rotating head; and
a curved needle positioned between the rotating head and the gripper head via a needle carrier, the curved needle being directionally manipulated in accordance with movement of the rotating head.

20. The method according to claim 19, further comprising one or more links embedded within the body portion for enabling a push/pull action that causes the rotational movement of the rotating head;
wherein the gripper head and the rotating head enable stabilization and manipulation of the curved needle relative to the body portion, the manipulation of the curved needle including at least griping, rotating, and/or articulating the needle.
